SEQUENCE LISTING (5 sequences)

SEQ ID NO: 1 - DNA, 1483 bp, Rhodococcus rhodochrous
  CDS (286)..(1386)
cgatcgaaccagcaacggggacgcacagtcgacgtagacctcgacctatccgccgttccgcagaaggacaccgaccaccaccacttcaacatccttcaacgtgcccggccagtccttcgacgaatcgaaacggcgaagagccgcctcggaccccccggccgaaccgctcgatgaactcccctacacgggtggcgcagaatgccaggacccgtgtcattccacgtcaattcacgcgccttttcacctcgtactgtcctgccaaacacaagcaacggaggtacggacatggtcgaatacacaaacacattcaaagttgctgcggtgcaggcacagcctgtgtggttcgacgcggccaaaacggtcgacaagaccgtgtccatcatcgcggaagcagcccggaacgggtgcgagctcgttgcgtttcccgaggtattcatcccggggtacccgtaccacatctgggtcgacagcccgctcgccggaatggcgaagttcgccgtgcgctaccacgagaattccctgacgatggacagcccgcacgtacagcggttgctcgatgccgcccgcgaccacaacatcgccgtagtggtgggaatcagcgagcgggatggcggcagcttgtacatgacccagctcatcatcgacgccgatgggcaactggtcgcccgacgccgcaagctcaagcccacccacgtcgagcgttcggtatacggagaaggaaacggctcggatatctccgtgtacgacatgcctttcgcacggcttggcgcgctcaactgctgggagcatttccagacgctcaccaagtacgcaatgtactcgatgcacgagcaggtgcacgtcgcgagctggcctggcatgtcgctgtaccagccggaggtccccgcattcggtgtcgatgcccagctcacggccacgcgtatgtacgcactcgagggacaaaccttcgtggtctgcaccacccaggtggtcacaccggaggcccacgagttcttctgcgagaacgaggaacagcgaaagttgatcggccgaggcggaggtttcgcgcgcatcatcgggcccgacggccgcgatctcgcaactcctctcgccgaagatgaggaggggatcctctacgccgacatcgatctgtctgcgatcaccttggcgaagcaggccgctgaccccgtgggccactactcacggccggatgtgctgtcgctgaacttcaaccagcgccgcaccacgcccgtcaacaccccactttccaccatccatgccacgcacacgttcgtgccgcagttcggggcactcgacggcgtccgtgagctcaacggagcggacgaacagcgcgcattgccctccacacattccgacgagacggaccgggcgacagccaccctctgactcgggcgcacccgtggcgcctccgaagcgccacgggtgtgtgaaggggcgagacaggggaatcggaggatcaccgagtacaacgcatcgtcgatcg

SEQ ID NO: 2 - protein, 366 aa, Rhodococcus rhodochrous
MVEYTNTFKVAAVQAQPVWFDAAKTVDKTVSIIAEAARNGCELVAFPEVFIPGYPYHIWVDSPLAGMAKFAVRYHENSLTMDSPHVQRLLDAARDHNIAVVVGISERDGGSLYMTQLIIDADGQLVARRRKLKPTHVERSVYGEGNGSDISVYDMPFARLGALNCWEHFQTLTKYAMYSMHEQVHVASWPGMSLYQPEVPAFGVDAQLTATRMYALEGQTFVVCTTQVVTPEAHEFFCENEEQRKLIGRGGGFARIIGPDGRDLATPLAEDEEGILYADIDLSAITLAKQAADPVGHYSRPDVLSLNFNQRRTTPVNTPLSTIHATHTFVPQFGALDGVRELNGADEQRALPSTHSDETDRATATL

SEQ ID NO: 3 - DNA, 20 bp, Artificial sequence
  Oligonucleotide probe
tggaaagtgcttcccaagca

SEQ ID NO: 4 - DNA, 29 bp, Artificial sequence
  Primer for gene amplification
tatatatcatatggtcgaatacacaaaca

SEQ ID NO: 5 - DNA, 28 bp, Artificial sequence
  Primer for gene amplification
taattaagcttcagagggtggctgtcgc

(19) United States
(12) Patent Application Publication
(10) Pub. No.: US 2003/0157672 A1
(43) Pub. Date: Aug. 21, 2003

(54) Nitrilase from rhodococcus rhodochrous ncimb 11216

(76) Inventors: Marion Ress-Loschke, Dossenheim (DE); Bernhard Hauer, Fussgonheim (DE); Ralf Mattes, Stuttgart (DE); Dirk Engels, Walddorfhaslach (DE)

Correspondence Address: KEIL & WEINKAUF, 1350 CONNECTICUT AVENUE, N.W., WASHINGTON, DC, 20036, US

(21) Appl. No.: 10/220,564

(22) PCT Filed: Feb. 27, 2001
(86) PCT No.: PCT/EP01/02191

(30) Foreign Application Priority Data
Mar. 3, 2000 (DE) 10010149.6

(51) Int. Cl. C12P 7/40; C12N 9/16; C07H 21/04; C12P 21/02; C12N 1/21; C12N 15/74

(52) U.S. Cl. 435/136; 435/69.1; 435/196; 435/252.3; 435/320.1; 536/23.2

(57) ABSTRACT

The invention relates to nucleic acid sequences which code for a polypeptide having nitrilase activity, to nucleic acid constructs comprising the nucleic acid sequences, and to vectors comprising the nucleic acid sequences or the nucleic acid constructs. The invention further relates to amino acid sequences which are encoded by the nucleic acid sequences, and to microorganisms comprising the nucleic acid sequences, the nucleic acid constructs or vectors comprising the nucleic acid sequences or the nucleic acid constructs.

The invention additionally relates to an enzymatic process for preparing carboxylic acids from the corresponding nitrites.

The invention relates to nucleic acid sequences which code for a polypeptide having nitrilase activity, to nucleic acid constructs comprising the nucleic acid sequences, and to vectors comprising the nucleic acid sequences or the nucleic acid constructs. The invention further relates to amino acid sequences which are encoded by the nucleic acid sequences, and to microorganisms comprising the nucleic acid sequences, the nucleic acid constructs or vectors comprising the nucleic acid sequences or the nucleic acid constructs.

The invention additionally relates to an enzymatic process for preparing carboxylic acids from the corresponding nitrites.

Aliphatic, aromatic and heteroaromatic carboxylic acids are compounds in demand for organic chemical synthesis. They are starting materials for a large number of active pharmaceutical ingredients or active ingredients for crop protection.

Various different synthetic routes to achiral or chiral carboxylic acids are disclosed in the literature. Thus, for example, optically active amino acids are obtained industrially by fermentation processes. These entail the disadvantage that a specific process must be developed for each amino acid. This is why chemical or enzymatic processes are used in order to be able to prepare a maximally wide range of different compounds. A disadvantage of chemical processes is that the stereocenter usually has to be constructed in complicated, multistage, not widely applicable synthesis [sic].

The enzymatic synthesis of chiral carboxylic acids is to be found in a number of patents or patent applications. WO92/05275 describes the synthesis of enantiomeric α-hydroxy-α-alkyl- or α-alkylcarboxylic acids in the presence of biological materials. Further syntheses of optically active α-substituted organic acids with microorganisms are described in EP-B-0 348 901, EP-B-0 332 379, EP-A-0 348 901 or its U.S. equivalent U.S. Pat. No. 5,283,193, EP-A-0 449 648, EP-B-0 473 328, EP-B-0 527 553 or its U.S. equivalent U.S. Pat. No. 5,296,373, EP-A-0 610 048, EP-A-0 610 049, EP-A-0 666 320 or WO 97/32030.

The biotechnological synthesis of achiral carboxylic acids with microorganisms is described, for example, in EP-A-0 187 680, EP-A-0 229 042, WO 89/00193, JP 08173152, JP 06153968, FR 2694571, EP-A0 502 476, EP-A-0 444 640 or EP-A-0 319 344.

A disadvantage of these processes is that they often lead to products with only low optical purity and/or that they proceed with only low space-time yields. This leads to economically unattractive processes. An additional disadvantage is that the enzymes present in the microorganisms used for synthesizing the achiral or chiral carboxylic acids usually have only a restricted substrate range, that is to say a microorganism always converts only particular aliphatic, aromatic or heteroaromatic nitrites. Specifically, aromatic and heteroaromatic nitrites such as, for example, cyanothiophenes or benzonitrile are converted poorly or not at all into the corresponding carboxylic acids.

It is an object of the present invention to develop further enzymes for preparing achiral and/or chiral carboxylic acids which can be used in a process for preparing achiral and/or chiral carboxylic acids which does not have the abovementioned disadvantages and specifically makes aromatic and/or heteroaromatic carboxylic acids available from the corresponding nitrites.

We have found that this object is achieved by the nucleic acid sequence isolated according-to the invention, which codes for a polypeptide having nitrilase activity, selected from the group of:

a) a nucleic acid sequence having the sequence depicted in SEQ ID NO: 1,

b) nucleic acid sequences which are derived from the nucleic acid sequence depicted in SEQ ID NO: 1 as a result of the degeneracy of the genetic code,

c) derivatives of the nucleic acid sequence depicted in SEQ ID NO: 1, which code for polypeptides having the amino acid sequences depicted in SEQ ID NO: 2 and have at least 95% homology at the amino acid level, with negligible reduction in the enzymatic action of the polypeptides.

Homologs of the nucleic acid sequence according to the invention with sequence SEQ ID NO: 1 mean, for example, allelic variants which have at least 95% homology at the derived amino acid level, advantageously at least 97% homology, preferably at least 98%, very particularly preferably at least 99% homology, over the entire sequence range. It is possible and advantageous for the homologies to be higher over regions forming part of the sequences. The amino acid sequence derived from SEQ ID NO: 1 is to be seen in SEQ ID NO: 2. Allelic variants comprise, in particular, functional variants which are obtainable by deletion, insertion or substitution of nucleotides from the sequence depicted in SEQ ID NO: 1, but with a negligible reduction in the enzymatic activity of the derived synthesized proteins. A negligible reduction in the enzymatic activity means an enzymatic activity which is advantageously at least 10%, preferably 30%, particularly preferaby 50%, very particularly preferably 70% of the enzymatic activity of the enzyme represented by SEQ ID NO: 2. The invention thus also relates to amino acid sequences which are encoded by the group of nucleic acid sequences described above. The invention advantageously relates to amino acid sequences encoded by sequence SEQ ID NO: 1.

Homologs of SEQ ID NO: 1 also mean, for example, fungal or bacterial homologs, truncated sequences, single-stranded DNA or RNA of the coding and noncoding DNA sequence. Homologs of SEQ ID NO: 1 have at the DNA level a homology of at least 60%, preferably of at least 70%, particularly preferably of at least 80%, very particularly preferably of at least 90%, over the entire DNA region indicated in SEQ ID NO: 1.

Homologs of SEQ ID NO: 1 additionally mean derivatives such as, for example, promoter variants. The promoters which precede the stated nucleotide sequences can be modified by one or more nucleotide exchanges, by insertion(s) and/or deletion(s) without, however, adversely affecting the functionality or effectiveness of the promoters. The promoters may moreover have their effectiveness increased by modifying their sequence or be completely replaced by more effective promoters even from organisms of different species.

Derivatives also mean variants whose nucleotide sequence in the region from −1 to −200 in front of the start codon or 0 to 1000 base pairs after the stop codon has been modified in such a way that gene expression and/or protein expression is altered, preferably increased.

SEQ ID NO: 1 or its homologs can advantageously be isolated by methods known to the skilled worker from bacteria, advantageously from Gram-positive bacteria, preferably from bacteria of the genera Nocardia, Rhodococcus, Streptomyces, Mycobacterium, Corynebacterium, Micrococcus, Proactinomyces or Bacillus, particularly preferably from bacteria of the genus Rhodococcus, Mycobacterium or Nocardia, very particularly preferably from the genus and species Rhodococcus sp., Rhodococcus rhodochrous, Nocardia rhodochrous or Mycobacterium rhodochrous.

SEQ ID No: 1 or its homologs or parts of these sequences can be isolated from other fungi or bacteria for example using conventional hybridization processes or the PCR technique. These DNA sequences hybridize under standard conditions with the sequences according to the invention. The hybridization is advantageously carried out with short oligonucleotides of the conserved regions, for example from the active center, and these can be identified in a manner known to the skilled worker by comparisons with other nitrilases or nitrile hydratases. However, it is also possible to use longer fragments of the nucleic acids according to the invention or the complete sequences for the hybridization. These standard conditions vary depending on the nucleic acid used, whether oligonucleotide, longer fragment or complete sequence, or depending on which type of nucleic acid, DNA or RNA, is used for the hybridization. Thus, for example, the melting temperatures of DNA:DNA hybrids are about 10° C. lower than those of DNA:RNA hybrids of the same length.

Standard conditions mean, for example, depending on the nucleic acid, temperatures between 42 and 58° C. in an aqueous buffer solution with a concentration between 0.1 and 5×SSC (1×SSC=0.15 M NaCl, 15 mM sodium citrate, pH 7.2) or additionally in the presence of 50% formamide, such as, for example, 42° C. in 5×SSC, 50% formamide. The hybridization conditions for DNA:DNA hybrids advantageously comprise 0.1×SSC and temperatures between about 20° C. and 45° C., preferably between about 30° C. and 45° C. The hybridization conditions for DNA:RNA hybrids preferably comprise 0.1×SSC and temperatures between about 30° C. and 55° C., preferably between about 45° C. and 55° C. These temperatures stated for the hybridization are melting temperatures calculated by way of example for a nucleic acid with a length of about 100 nucleotides and a G+C content of 50% in the absence of formamide. The experimental conditions for the DNA hybridization are described in relevant textbooks of genetics such as, for example, Sambrook et al., “Molecular Cloning”, Cold Spring Harbor Laboratory, 1989, and can be calculated by formulae known to the skilled worker, for example depending on the length of the nucleic acids, the nature of the hybrids or the G+C content. The skilled worker can find further information on hybridization in the following textbooks: Ausubel et al. (eds), 1985, Current Protocols in Molecular Biology, John Wiley & Sons, New York; Hames and Higgins (eds), 1985, Nucleic,Acids Hybridization: A Practical Approach, IRL Press at Oxford University Press, Oxford:; Brown (ed), 1991, Essential Molecular Biology: A Practical Approach, IRL. Press at Oxford University Press, Oxford.

The nucleic acid construct according to the invention means the nitrilase gene of sequence SEQ ID No. 1 and its homologs, which have advantageously been functionally linked to one or more regulatory signals to increase gene expression. These regulatory sequences are, for example, sequences to which the inducers or repressors bind and thus regulate the expression of the nucleic acid. In addition to these novel regulatory sequences, it is also possible for the natural regulation of these sequences to be present in front of the actual structural genes and, where appropriate, to have been genetically modified so that the natural regulation is switched off and the expression of the genes has been increased. The nucleic acid construct may, however, also have a simpler structure, that is to say no additional regulatory signals have been inserted in front of the sequence SEQ ID No. 1 or its homologs, and the natural promoter with its regulation has not been deleted. Instead, the natural regulatory sequence is mutated in such a way that the regulation no longer takes place, and gene expression is increased. The nucleic acid construct may additionally advantageously comprise one or more enhancer sequences, functionally linked to the promoter, which make increased expression of the nucleic acid sequence possible. It is also possible to insert advantageous additional sequences at the 3′ end of the DNA sequences, such as other regulatory elements or terminators. The nucleic acids according to the invention may be present in one or more copies in the construct. The construct may also comprise further markers such as antibiotic resistances or auxotrophy-complementing genes where appropriate for selection of the construct.

Advantageous regulatory sequences for the process according to the invention are, for example, present in promoters such as cos, tac, trp, tet, trp-tet, lpp, lac, lpp-lac, lacI^{q}, T7, T5, T3, gal, trc, ara, SP6, λ-P_{R} or the λ-P_{L} promoter, which are advantageously used in Gram-negative bacteria. Further advantageous regulatory sequences are in, for example, the Gram-positive promoters amy and SPO2, in the fungal or yeast promoters ADC1, MFα, AC, P-60, CYC1, GAPDH, TEF, rp28, ADH. Also advantageous in this connection are the promoters of pyruvate decarboxylase and of methanol oxidase from, for example, Hansenula. It is also possible to use artificial promoters for the regulation.

The nucleic acid construct is advantageously inserted into a vector such as, for example, a plasmid, a phage or other DNA for expression in a host organism, which makes optimal expression of the genes in the host possible. These vectors represent a further development of the invention. Examples of suitable plasmids in E. coli are pLG338, pACYC184, pBR322, pUC18, pUC19, pKC30, pRep4, pHS1, pHS2, pPLc236, pMBL24, pLG200, pUR290, pIN-III¹¹³-B1, λgt11 or pBdCI, in Streptomyces are pIJ101, pIJ364, pIJ702 or pIJ361, in Bacillus are pUB110, pC194 or pBD214, in Corynebacterium are pSA77 or pAJ667, in fungi are pALS1, pIL2 or pBB116, in yeasts are 2 μM, pAG-1, YEp6, YEp13 or pEMBLYe23 or in plants are pLGV23, pGHlac⁺, pBIN19, pAK2004 or pDH51. Said plasmids represent a small selection of the possible plasmids. Further plasmids are well known to the skilled worker and can be found, for example, in the book Cloning Vectors (eds. Pouwels P. H. et al. Elsevier, Amsterdam-New York-Oxford, 1985, ISBN 0 444 904018).

The nucleic acid construct advantageously also contains, for expression of the other genes present, in addition 3′ and/or 5′ terminal regulatory sequences to increase expression, which are selected for optimal expression depending on the selected host organism and gene or genes.

These regulatory sequences are intended to make specific expression of the genes and protein expression possible. This may mean, for example, depending on the host organism, that the gene is expressed or overexpressed only after induction, or that it is immediately expressed and/or overexpressed.

The regulatory sequences or factors may moreover preferably influence positively, and thus increase, expression of the introduced genes. Thus, enhancement of the regulatory elements can take place advantageously at the level of transcription, by using strong transcription signals such as promoters and/or enhancers. However, it is also possible in addition to enhance translation by, for example, improving the stability of the mRNA.

In another embodiment of the vector, the vector comprising the nucleic acid construct according to the invention or the nucleic acid according to the invention can also advantageously be introduced in the form of a linear DNA into the microorganisms and be integrated by heterologous or homologous recombination into the genome of the host organism. This linear DNA may consist of a linearized vector such as a plasmid or only of the nucleic acid construct or of the nucleic acid.

For optimal expression of heterologous genes in organisms, it is advantageous to modify the nucleic acid sequences to accord with the codon usage specifically used in the organism. The codon usage can easily be established on the basis of computer analyses of other known genes in the relevant organism.

Suitable host organisms for the nucleic acid according to the invention or the nucleic acid construct are in principle all procaryotic or eucaryotic organisms. The host organisms advantageously used are microorganisms such as bacteria, fungi or yeasts. It is advantageous to use Gram-positive or Gram-negative bacteria, preferably bacteria of the family Enterobacteriaceae, Pseudomonadaceae, Streptomycetaceae, Mycobacteriaceae, or Nocardiaceae, particularly preferably bacteria of the genera Escherichia, Pseudomonas, Nocardia, Mycobacterium, Streptomyces oder Rhodococcus. Very particular preference is given to the genus and species Escherichia coli, Rhodococcus rhodochrous, Nocardia rhodochrous, Mycobacterium rhodochrous or Streptomyces lividans.

The host organism according to the invention moreover preferably comprises at least one proteinaceous agent for folding the polypeptides it has synthesized and, in particular, the nucleic acid sequences having nitrilase activity described in this invention and/or the genes encoding this agent, the amount of this agent present being greater than that corresponding to the basic amount in the microorganism considered. The genes coding for this agent are present in the chromosome or in extrachromosomal elements such as, for example, plasmids.

The invention further relates to a process for preparing chiral or achiral carboxylic acids, which comprise converting nitriles in the presence of an amino acid sequence encoded by the nucleic acids according to the invention, or a growing, dormant or disrupted abovementioned microorganism (=host organism) which contains either a nucleic acid sequence according to the invention, a nucleic acid construct according to the invention which contains a nucleic acid according to the invention linked to one or more regulatory signals, or a vector according to the invention, into the chiral or achiral carboxylic acids.

An advantageous embodiment of the process is the conversion of chiral or achiral aliphatic nitriles into the corresponding carboxylic acids.

Another preferred embodiment of the process is a process for preparing chiral or achiral carboxylic acids, wherein nitrites of the general formula I

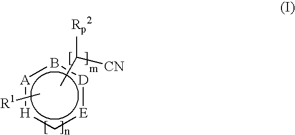

are converted in the presence of an amino acid sequence encoded by the nucleic acids according to the invention, or a growing, dormant or disrupted abovementioned microorganism which contains either a nucleic acid sequence according to the invention, a nucleic acid construct according to the invention which contains a nucleic acid according to the invention linked to one or more regulatory signals, or a vector according to the invention, into carboxylic acids of the general formula II

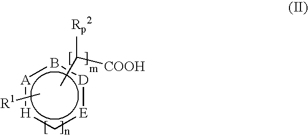

where the substituents and variables in the formulae I and II have the following meanings:

n=0 or 1

m=0, 1, 2 or 3, where for m>2 there is one or no double bond present between two adjacent carbon atoms,

p=0 or 1

A, B, D and E independently of one another are CH, N or CR³

H=O, S, NR⁴, CH or CR³, when n=0, or CH, N or CR³, when n=1,

it being possible for two adjacent variables A, B, D, E or H together to form another substituted or unsubstituted aromatic, saturated or partially saturated ring with 5 to 8 atoms in the ring which may contain one or more heteroatoms such as O, N or S, and not more than three of the variables A, B, D, E or H being a heteroatom,

R¹ is hydrogen, substituted or unsubstituted, branched or unbranched C₁-C₁₀-alkyl or C₁-C₁₀-alkoxy, substituted or unsubstituted aryl or hetaryl, hydroxyl, halogen, C₁-C₁₀-alkylamino or amino,

R² is hydrogen, substituted or unsubstituted, branched or unbranched C₁-C₁₀-alkyl or C₁-C₁₀-alkoxy, substituted or unsubstituted aryl or hetaryl, hydroxyl, C₁-C₁₀-alkylamino or amino,

R³ is hydrogen, substituted or unsubstituted, branched or unbranched C₁-C₁₀-alkyl or C₁-C₁₀-alkoxy, substituted or unsubstituted aryl, hetaryl, hydroxyl, halogen, C₁-C₁₀-alkylamino or amino,

R⁴ is hydrogen, substituted or unsubstituted, branched or unbranched C₁-C₁₀-alkyl.

R¹ in the compounds of the formulae I and II is hydrogen, substituted or unsubstituted, branched or unbranched C₁-C₁₀-alkyl or C₁-C₁₀-alkoxy, substituted or unsubstituted aryl or hetaryl, hydroxyl, halogen such as fluorine, chlorine or bromine, C₁-C₁₀-alkylamino or amino.

Alkyl radicals which may be mentioned are substituted or unsubstituted, branched or unbranched C₁-C₁₀-alkyl chains such as, for example, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, n-nonyl or n-decyl. Methyl, ethyl, n-propyl, n-butyl, i-propyl or i-butyl is preferred.

Alkoxy radicals which may be mentioned are substituted or unsubstituted, branched or unbranched C₁-C₁₀-alkoxy chains such as, for example, methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy., 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-Ethyl-1-methylpropoxy, 1-ethyl-2-methylpropoxy, hexyloxy, heptyloxy, octyloxy, nonyloxy or decyloxy and the branched-chain homologs thereof.

Aryl radicals which may be mentioned are substituted and unsubstituted aryl radicals which contain 6 to 20 carbon atoms in the ring or ring system. These may comprise aromatic rings fused together or aromatic rings linked by alkyl, alkylcarbonyl, alkenyl or alkenylcarbonyl chains, carbonyl, oxygen or nitrogen. The aryl radicals may also be linked, where appropriate, via a C₁-C₁₀-alkyl, C₃-C₈-alkenyl, C₃-C₆-alkynyl or C₃-C₈-cycloalkyl chain to the basic framework. Phenyl or naphthyl is preferred.

Hetaryl systems which may be mentioned are substituted or unsubstituted, simple or fused aromatic ring systems with one or more heteroaromatic 3- to 7-membered rings which may contain one or more heteroatoms such as N, O or S and may, where appropriate, be linked via a C₁-C₁₀-alkyl, C₃-C₈-alkenyl or C₃-C₈-cycloalkyl chain to the basic framework. Examples of such hetaryl radicals are pyrazole, imidazole, oxazole, isoxazole, thiazole, triazole, pyridine, quinoline, isoquinoline, acridine, pyrimidine, pyridazine, pyrazine, phenazine, purine or pteridine. The hetaryl radicals may be linked via the heteroatoms or via the various carbon atoms in the ring or ring system or via the substituents to the basic framework. Pyridine, imidazole, pyrimidine, purine, pyrazine or quinoline is preferred.

Alkylamino radicals which may be mentioned are substituted or unsubstituted, branched or unbranched C₁-C₁₀-alkylamino chains such as, for example, methylamino, ethylamino, n-propylamino, 1-methylethylamino, n-butylamino, 1-methylpropylaminoamino [sic], 2-methylpropylamino, 1,1-dimethylethylamino, n-pentylamino, 1-methylbutylamino, 2-methylbutylamino, 3-methylbutylamino, 2,2-dimethylpropylamino, 1-ethylpropylamino, n-hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino, 1-ethyl-2-methylpropylamino, n-heptylamino, n-octylamino, n-nonylamino or n-decylamino. Methylamino, ethylamino, n-propylamino, n-butylamino, i-propylamino or i-butylamino is preferred.

Suitable substituents for said R¹ radicals are, for example, one or more substituents such as halogen such as fluorine, chlorine or bromine, thio [sic], cyano, nitro, amino, hydroxyl, alkyl, alkoxy, alkenyl, alkenyloxy, alkynyl or other aromatic or other saturated or unsaturated nonaromatic rings or ring systems. Preference is given to alkyl radicals such as C₁-C₆-alkyl such as methyl, ethyl, propyl or butyl, aryl such as phenyl, halogen such as chlorine, fluorine or bromine, hydroxyl or amino.

R² in the compounds of the formulae I and II is hydrogen, such as substituted or unsubstituted, branched or unbranched C₁-C₁₀-alkyl or C₁-C₁₀-alkoxy, substituted or unsubstituted aryl or hetaryl, hydroxyl, C₁-C₁₀-alkylamino or amino.

Alkyl radicals which may be mentioned are substituted or unsubstituted, branched or unbranched C₁-C₁₀-alkyl chains such as, for example, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, n-nonyl or n-decyl. Methyl, ethyl, n-propyl, n-butyl, i-propyl or i-butyl is preferred.

Alkoxy radicals which may be mentioned are substituted or unsubstituted, branched or unbranched C₁-C₁₀-alkoxy chains such as, for example, methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-Ethyl-1-methylpropoxy, 1-ethyl-2-methylpropoxy, hexyloxy, heptyloxy, octyloxy, nonyloxy or decyloxy and the branched-chain homologs thereof.

Aryl radicals which may be mentioned are substituted and unsubstituted aryl radicals which contain 6 to 20 carbon atoms in the ring or ring system. These may comprise aromatic rings fused together or aromatic rings linked by alkyl, alkylcarbonyl, alkenyl or alkenylcarbonyl chains, carbonyl, oxygen or nitrogen. The aryl radicals may also be linked, where appropriate, via a C₁-C₁₀-alkyl, C₃-C₈-alkenyl, C₃-C₆-alkynyl or C₃-C₈-cycloalkyl chain to the basic framework. Phenyl or naphthyl is preferred.

Hetaryl systems which may be mentioned are substituted or unsubstituted, simple or fused aromatic ring systems with one or more heteroaromatic 3- to 7-membered rings which may contain one or more heteroatoms such as N, O or S and may, where appropriate, be linked via a C₁-C₁₀-alkyl, C₃-C₈-alkenyl or C₃-C₈-cycloalkyl chain to the basic framework. Examples of such hetaryl radicals are pyrazole, imidazole, oxazole, isoxazole, thiazole, triazole, pyridine, quinoline, isoquinoline, acridine, pyrimidine, pyridazine, pyrazine, phenazine, purine or pteridine. The hetaryl radicals may be linked via the heteroatoms or via the various carbon atoms in the ring or ring system or via the substituents to the basic framework. Pyridine, imidazole, pyrimidine, purine, pyrazine or quinoline is preferred.

Alkylamino radicals which may be mentioned are substituted or unsubstituted, branched or unbranched C₁-C₁₀-alkylamino chains such as, for example, methylamino, ethylamino, n-propylamino, 1-methylethylamino, n-butylamino, 1-methylpropylaminoamino [sic], 2-methylpropylamino, 1,1-dimethylethylamino, n-pentylamino, 1-methylbutylamino, 2-methylbutylamino, 3-methylbutylamino, 2,2-dimethylpropylamino, 1-ethylpropylamino, n-hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino, 1-ethyl-2-methylpropylamino, n-heptylamino, n-octylamino, n-nonylamino or n-decylamino. Methylamino, ethylamino, n-propylamino, n-butylamino, i-propylamino or i-butylamino is preferred. Suitable substituents for said R² radicals are, for example, one or more substituents such as halogen such as fluorine, chlorine or bromine, thio [sic], nitro, amino, hydroxyl, alkyl, alkoxy, alkenyl, alkenyloxy, alkynyl or other aromatic or other saturated or unsaturated nonaromatic rings or ring systems. Preference is given to alkyl radicals such as C₁-C₆-alkyl such as methyl, ethyl, propyl or butyl, aryl such as phenyl, halogen such as chlorine, fluorine or bromine, hydroxyl or amino.

R³ in the compounds of the formula I and II is hydrogen, substituted or unsubstituted, branched or unbranched C₁-C₁₀-alkyl or C₁-C₁₀-alkoxy, substituted or unsubstituted aryl or hetaryl, hydroxyl, halogen, such as fluorine, chlorine or bromine, C₁-C₁₀-alkylamino or amino.

Alkyl radicals which may be mentioned are substituted or unsubstituted, branched or unbranched C₁-C₁₀-alkyl chains such as, for example, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, n-nonyl or n-decyl. Methyl, ethyl, n-propyl, n-butyl, i-propyl or i-butyl is preferred.

Alkoxy radicals which may be mentioned are substituted or unsubstituted, branched or unbranched C₁-C₁₀-alkoxy chains such as, for example, methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-Ethyl-1-methylpropoxy, 1-ethyl-2-methylpropoxy, hexyloxy, heptyloxy, octyloxy, nonyloxy or decyloxy and the branched-chain homologs thereof.

Aryl radicals which may be mentioned are substituted and unsubstituted aryl radicals which contain 6 to 20 carbon atoms in the ring or ring system. These may comprise aromatic rings fused together or aromatic rings linked by alkyl, alkylcarbonyl, alkenyl or alkenylcarbonyl chains, carbonyl, oxygen or nitrogen. The aryl radicals may also be linked, where appropriate, via a C₁-C₁₀-alkyl, C₃-C₈-alkenyl, C₃-C₆-alkynyl or C₃-C₈-cycloalkyl chain to the basic framework. Phenyl or naphthyl is preferred.

Hetaryl systems which may be mentioned are substituted or unsubstituted, simple or fused aromatic ring systems with one or more heteroaromatic 3- to 7-membered rings which may contain one or more heteroatoms such as N, O or S and may, where appropriate, be linked via a C₁-C₁₀-alkyl, C₃-C₈-alkenyl or C₃-C₈-cycloalkyl chain to the basic framework. Examples of such hetaryl radicals are pyrazole, imidazole, oxazole, isoxazole, thiazole, triazole, pyridine, quinoline, isoquinoline, acridine, pyrimidine, pyridazine, pyrazine, phenazine, purine or pteridine. The hetaryl radicals may be linked via the heteroatoms or via the various carbon atoms in the ring or ring system or via the substituents to the basic framework. Pyridine, imidazole, pyrimidine, purine, pyrazine or quinoline is preferred.

Alkylamino radicals which may be mentioned are substituted or unsubstituted, branched or unbranched C₁-C₁₀-alkylamino chains such as, for example, methylamino, ethylamino, n-propylamino, 1-methylethylamino, n-butylamino, 1-methylpropylaminoamino [sic], 2-methylpropylamino, 1,1-dimethylethylamino, n-pentylamino, 1-methylbutylamino, 2-methylbutylamino, 3-methylbutylamino, 2,2-dimethylpropylamino, 1-ethylpropylamino, n-hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino, 1-ethyl-2-methylpropylamino, n-heptylamino, n-octylamino, n-nonylamino or n-decylamino. Methylamino, ethylamino, n-propylamino, n-butylamino, i-propylamino or i-butylamino is preferred.

Suitable substituents for said R³ radicals are, for example, one or more substituents such as halogen such as fluorine, chlorine or bromine, thio [sic], nitro, amino, hydroxyl, alkyl, alkoxy, alkenyl, alkenyloxy, alkynyl or other aromatic or other saturated or unsaturated nonaromatic rings or ring systems. Preference is given to alkyl radicals such as C₁-C₆-alkyl such as methyl, ethyl, propyl or butyl, aryl such as phenyl, halogen such as chlorine, fluorine or bromine, hydroxyl or amino.

R⁴ in the compounds of the formulae I and II is hydrogen or substituted or unsubstituted, branched or unbranched C₁-C₁₀-alkyl.

Alkyl radicals which may be mentioned are substituted or unsubstituted, branched or unbranched C₁-C₁₀-alkyl chains such as, for example, methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, n-nonyl or n-decyl. Methyl, ethyl, n-propyl, n-butyl, i-propyl or i-butyl is preferred.

Suitable substituents for said R⁴ radicals are, for example, one or more substituents such as halogen such as fluorine, chlorine or bromine, thio [sic], nitro, amino, hydroxyl, alkyl, alkoxy, alkenyl, alkenyloxy, alkynyl or other aromatic or other saturated or unsaturated nonaromatic rings or ring systems. Preference is given to alkyl radicals such as C₁-C₆-alkyl such as methyl, ethyl, propyl or butyl, aryl such as phenyl, halogen such as chlorine, fluorine or bromine, hydroxyl or amino.

It is also possible and advantageous to convert aromatic or aliphatic, saturated or unsaturated dinitriles in the process according to the invention.

The process according to the invention is advantageously carried out at a pH of from 4 to 11, preferably from 4 to 9.

In addition, it is advantageous to use in the process from 0.01 to 10% by weight, preferably 0.1 to 10% by weight, particularly preferably 0.5 to 5% by weight, of nitrile. Different amounts of nitrile can be used in the reaction depending on the nitrile. The smallest amounts (equals amounts between 0.01 to [sic] 5% by weight) of nitrile are advantageously used in the case of nitriles (cyanohydrins) which are in equilibrium with the corresponding aldehydes and hydrocyanic acid. [sic] Since the aldehyde is usually toxic for the microorganisms or enzymes.

Volatile nitriles are likewise advantageously employed in amounts between 0.01 to [sic] 5% by weight. With larger amounts of cyanohydrin or nitrile the reaction is retarded. In the case of nitrites which have only slight or virtually no solvent properties, or nitriles which dissolve in only very small amounts in aqueous medium, it is also possible and advantageous to employ larger amounts than those stated above. For increasing the conversion and the yield it is advantageous to carry out the reaction with continuous addition-of the nitrile. The product can be isolated after the end of the reaction or else be removed continuously in a bypass.

The process according to the invention is advantageously carried out at a temperature between 0° C. to [sic] 80° C., preferably between 10° C. to [sic] 60° C., particularly preferably between 15° C. to [sic] 50° C.

It is advantageous to mention in the process according to the invention aromatic or heteroaromatic nitrites such as 2-phenylpropionitrile, 2-hydroxy-phenylactonitrile [sic], 2-amino-2-phenylacetonitrile, benzonitrile, phenylacetonitrile, trans-cinnamonitrile, 3-cyanothiophene or 3-cyanomethylthiophene.

Chiral nitrites in the process according to the invention mean nitrites which consist of a 50:50 mixture of the two enantiomers or of any other mixture with enrichment of one of the two enantiomers in the mixture. Examples which may be mentioned of such nitrites are 2-phenylpropionitrile, 2-hydroxy-phenylacetonitrile [sic], 2-amino-2-phenylacetonitrile, 2-chloropropionitrile or 2-hydroxypropionitrile.

Chiral carboxylic acids in the process according to the invention mean those showing an enantiomeric enrichment. The process preferably results in enantiomeric purities of at least 90% ee, preferably of at least 95% ee, particularly preferably of at least 98% ee, very particularly preferably at least 99% ee.

The process according to the invention makes it possible to convert a large number of chiral or achiral nitrites into the corresponding chiral or achiral carboxylic acids. It is possible in the process to convert at least 25 mmol of nitrile/h×mg of protein or at least 25 mmol of nitrile/h×g dry weight of the microorganisms, preferably at least 30 mmol of nitrile/h×mg of protein or at least 30 mmol of nitrile/h×g dry weight, particularly preferably at least 40 mmol of nitrile/h×mg of protein or at least 40 mmol of nitrile/h×g dry weight, very particularly preferably at least 50 mmol of nitrile/h×mg of protein or fat least 50 mmol of nitrile/h×g dry weight.

It is possible to use growing cells which comprise the nucleic acids, nucleic acid constructs or vectors according to the invention for the process according to the invention. Dormant or disrupted cells can also be used. Disrupted cells mean, for example, cells which have been made permeable by a treatment. with, for example, solvents, or cells which have been disintegrated by an enzyme treatment, by a mechanical treatment (e.g. French press or ultrasound) or by any other method. The crude extracts obtained in this way are suitable and advantageous for the process according to the invention. Purified or partially purified enzymes can also be used for the process. Immobilized microorganisms or enzymes are likewise suitable and can advantageously be used in the reaction.

The chiral or achiral carboxylic acids prepared in the process according to the invention can advantageously be isolated from the aqueous reaction solution by extraction or crystallization or by extraction and crystallization. For this purpose, the aqueous reaction solution is acidified with an acid such as a mineral acid (e.g. HCl or H₂SO₄) or an organic acid, advantageously to pH values below 2, and then extracted with an organic solvent. The extraction can be repeated several times to increase the yield. Organic solvents which can be used are in principle all solvents which show a phase boundary with water, where appropriate after addition of salts. Advantageous solvents are solvents such as toluene, benzene, hexane, methyl tert-butyl ether or ethyl acetate. The products can also be purified advantageously by binding to an ion exchanger and subsequently eluting with a mineral acid or carboxylic acid such as HCL [sic], H₂SO₄, formic acid or acetic acid.

After concentration of the aqueous or organic phase, the products can usually be isolated in good chemical purities, meaning a chemical purity of greater than 90%. After extraction, the organic phase with the product can, however, also be only partly concentrated, and the product can be crystallized. For this purpose, the solution is advantageously cooled to a temperature of from 0° C. to 10° C. The crystallization can also take place directly from the organic solution. The crystallized product can be taken up again in the same or a different solvent for renewed crystallization and be crystallized once again. The subsequent crystallization at least once may, depending on the position of the eutectic composition, further increase the enantiomeric purity of the product.

The chiral or achiral carboxylic acids can, however, also be crystallized out of the aqueous reaction solution immediately after acidification with an acid to a pH advantageously below 2. This advantageously entails the aqueous solution being concentrated by heating to reduce its volume by 10 to 90%, preferably 20 to 80%, particularly preferably 30 to 70%. The crystallization is preferably carried out with cooling. Temperatures between 0° C. and 10° C. are preferred for the crystallization. Direct crystallization from the aqueous solution is preferred for reasons of cost. It is likewise preferred to work up the chiral carboxylic acids via extraction and, where appropriate, subsequent crystallization.

With these preferred types of workup, the product of the process according to the invention can be isolated in yields of from 60 to 100%, preferably from 80 to 100%, particularly preferably from 90 to 100%, based on the nitrile employed for the reaction. The isolates [sic] product has a high chemical purity of >90%, preferably >95%, particularly preferably >98%. In addition, the product [sic] in the case of chiral nitrites and chiral carboxylic acids have high enantiomeric purity, which may be increased further by crystallization.

The products obtained in this way are suitable as starting material for organic syntheses to prepare drugs or agrochemicals or for racemate resolution.

### EXAMPLES

Isolation and Heterologous Expression of the nitA Gene from Rhodococcus fhodochrous NCIMB 11216

### Example 1

Isolation of the nitA Gene from Rhodococcus rhodochrous NCIMB 11216

The nitA gene was isolated from Rhodococcus rhodochrous NCIMB 11216 by isolating DNA from the cells, setting up a phage gene bank and screening the latter with an oligonucleotide probe.

1.1 Isolation of DNA from R.rhodochrous NCIMB 11216

To prepare genomic DNA from Rhodococcus rhodochrous NCIMB 11216 as described by Sambrook et al., 1989, 2×100 ml of overnight culture (in dYT medium, Sambrook, J., Fritsch, E. F. and Maniatis, T., 1989, Molecular cloning: a laboratory manual, 2nd edition, Cold Spring Harbor Laboratory Press. Cold Spring Harbor, N.Y.) were centrifuged, and the pellets were resuspended in 8 ml of 25 mM Tris/HCl, 25 mM EDTA, 10% sucrose (w/v), pH 8.0. Lysozyme treatment of the combined cultures at 37° C. for 15 min (addition of 2 ml of lysozyme, 100 mg/ml in 10 mM Tris/HCl, 0.1 mM EDTA, pH 8.0) was followed by addition of 2 ml of 10% (w/v) Na lauroyl sarcosinate and incubation at 65° C. for 15 min, mixing thoroughly several times. Then CsCl was added in a final concentration of 1 g/ml and dissolved at 65° C. and, after addition of ethidium bromide in a final concentration of 0.4 mg/ml, an ultracentrifugation was carried out in a fixed-angle rotor (Sorvall T1270, 83500 g, 48 h, 17° C.). The chromosomal DNA band was aspirated off under UV light, dialyzed against TE 10.1 (10 mM Tris/HCl, 1 mM EDTA, pH 8.0) for 2 h and extracted 3 times with phenol solution (saturated with 10 mM Tris/HCl, pH 8). Finally, the DNA was again dialyzed 3 times against TE 10.01 (10 mM Tris/HCl, 0.1 mM EDTA, pH 8.0), and stored at 4° C. This resulted in about 1.5 ml of DNA solution with a concentration of about 500 μg/ml.

1.2 Preparation of a phage gene bank from the DNA from R.rhodochrous NCIMB 11216

The vector used for the gene bank was the phage λ±RESIII: this substitution vector contains the lux operon as replacement fragment, which makes visual detection of the background possible by bioluminescence, and integrated res (“resolution”) sites from Tn1721 and the replication functions of pTW601-1, so that the vector can be transformed in a strain with appropriate transposase into an autonomously replicating plasmid (Altenbuchner, 1993, A new λ RES vector with a built-in Tn1721-encoded, excision system, Gene 123, 63-68).

1.2.1 Isolation of λ±RESIII-DNA (as described by Sambrook et al., 1989)

10¹⁰ cells were spun down from an overnight culture of E.coli TAP 90 (LB₀, Sambrook et al., 1989, and 10 mM MgSO₄, 0.2% maltose (w/v)) and the pellet was resuspended in 3 ml of SM phage buffer (50 mM Tris/HCl, 100 mM NaCl, 8 mM MgSO₄, 0.01% (w/v) gelatin). After infection with 1.5×10⁸−1.5×10⁹ plaque forming units (pfu) of λ RESIII phage lysate at 37° C. for 20 min, the mixture was added to 500 ml of LB₀, 10 mM MgSO₄, 0.2% maltose in a 2 l Erlenmeyer flask. A total of four such mixtures was stirred at 37° C. for 9 to 12 h until cell lysis was detectable. For complete lysis, 10 ml of chloroform were added to each flask and stirring was continued at 37° C. for 30 min. Cellular nucleic acids were digested by adding DNase and RNase (1 μg/ml of each) and stirring at room temperature for 30 min. Then 29.2 g of NaCl were added to each mixture and dissolved, the mixture was centrifuged at 8300 g for 10 min, and the supernatants were mixed with 10% PEG 6000. For the subsequent phage precipitation, the mixtures were stirred at 4° C. overnight and then centrifuged at 14000 g for 15 min. The pellets were dried and then each taken up in 5 ml of SM buffer, mixed with 5 ml of chloroform and centrifuged at 3000 g for 15 min. The aqueous phases with the phages were combined, mixed with 0.75 g/ml CsCl and, after dissolving was complete, centrifuged for 24 h (Sorvall T1270 fixed angle rotor, 98400 g, 48 h, 17° C.). The visible phage band was aspirated off and dialyzed 2×against 50 mM Tris/HCl, 10 mM NaCl, 10 MM MgCl₂, pH 8.0. Addition of 20 mM EDTA, 50 μg/ml proteinase K and 0.5% SDS was followed by incubation at 65° C. for 1 h. Extractions were then carried out 1× with phenol (saturated with 10 mM Tris/HCl, pH 8), 1× with phenol (saturated with 10 mM Tris/HCl, pH 8)/chloroform (50/50 v/v) and 1× with chloroform. The DNA was finally dialyzed 3× against TE 10.1 and 1× against TE 10.01, the titer was determined on E.coli TAP 90 (see 1.2.3 [sic]), and the λ±RESIII DNA was stored at 4° C.

1.2.2 Cloning of genomic DNA into λ±RESIII vectors

For cloning of genomic R.rhodochrous NCIMB 11216 DNA fragments, firstly the λ±RESIII arm fragments were prepared by digesting λ±RESIII DNA, 2 μg in a volume of 100 μl, with 20 U of BamHI at 37° C. for 5 h. After extraction with phenol (saturated with 10 mM Tris/HCl, pH 8)/chloroform (50/50 v/v), isopropanol precipitation and washing with 70% and 100% ethanol (precooled to −20° C.), the DNA was dissolved in TE 10.01 and then treated with 20 U of SalI (37° C. for 5 h). Phenol/chloroform extraction, isopropanol precipitation, washing and dissolving in TE 10.01 were repeated.

The genomic DNA fragments were prepared by partial digestion—after recording the kinetics for the enzyme batch used—of 10 μg of genomic DNA in 100 μl mixture with 0.5 U of Sau3AI for 5 min. After fractionation by electrophoresis on a 0.8% low melting point agarose gel, the fragment range from 8 to 14 kb was isolated and eluted from the gel as described by Parker & Seed (1980). The genomic DNA fragments were ligated with the λ±RESIII arms at 16° C. overnight.

The ligation mixtures were finally packaged in vitro using phage extracts which had previously been prepared from the “packaging extract donor” E.coli BHB 2688 (“freeze thaw lysate”, FTL, Sambrook et al., 1989) and the “prehead donor” E.coli BHB 2690 (“sonicated extract”, SE, Sambrook et al., 1989). For the packaging, 5 μl of ligation mixture, 7 μl of buffer A (20 mM Tris/HCl, 3 mM MgCl₂, 1 mM EDTA, 0.05% β-mercaptoethanol, pH 8.0), 7 μl of buffer Ml (6.7 mM Tris/HCl, 33 mM spermidine, 100 mM putrescine, 17.8 mM ATP,. 0.2% β-mercaptoethanol, 20 mM MgCl₂, pH 8), 15 μl of SE and 10 μl of FTL were mixed and incubated at room temperature for 1 h. Then 500 μl of SM buffer and 1 drop of chloroform were added and mixed, and the mixtures were centrifuged and stored at 4° C.

The titer of the phage gene bank prepared was determined by infecting the strain E.coli TAP 90 (Patterson & Dean, 1987). This was done by incubating logarithmically growing cells (cultured in LB₀, 10 mM MgCl₂, 0.5% maltose) with 100 μl of various dilutions of the packaging or phage lysate in SM buffer at 37° C. for 30 min. The mixtures were then each briefly mixed with 3 ml of top agar (0.8% of bacto agar, 10 mM MgCl₂, 0.5% maltose) equilibrated at 42° C., and layered onto LB₀ agar plates with 10 mM MgCl₂ (prewarmed to 37° C.). After incubation at 37° C. for 12-16 h, the plaques were counted to determine the titer. The titer of the gene bank prepared was about 4×10⁵ pfu/ml.

1.2.3 Conversion of the recombinant λ±RESIII phages into a plasmid

The resulting recombinant λ±RESIII phages were converted in the strain E.coli HB 101 F′ [::Tn1739lac], which harbors the transposon Tn1739 with the resolvase gene under the control of the tac promoter (Altenbuchner, 1993, see above), into an autonomously replicating plasmid. Before the infection, the strain was cultured in 5 ml of LB₀ with 10 mM MgCl₂ and 0.5% maltose until the OD₆₀₀ was 0.6 to 0.8 and 100 μl thereof were infected with a suitable amount of phage lysate at room temperature for 30 min. The mixture was roller cultured in 5 ml of prewarmed dYT, 1 mM isopropyl β-thiogalactopyranoside (IPTG) at 37° C. for 1 h, centrifuged and resuspended in the runback, and the cells were plated out on dYT agar plates with 100 μg/ml kanamycin and incubated at 37° C. overnight.

Cells whose converted λ±RESIII molecule still contains the original replacement fragment with the lux operon and thus contains no genomic insert (gene bank-background) were visualized by inducing the plates at 30° C. for 3 h and counting the bioluminescent cells in the dark. The gene bank background amounted to 13% according to the proportion of luminescent cells.

1.3 Screening of the nitrilase gene nitA from R.rhodochrous NCIMB 11216

Recombinant λ±RESIII phages containing chromosomal DNA fragments with the nitrilase gene from R.rhodochrous NCIMB 11216 were identified by hybridization of the phage plaques with the oligonucleotide probe

“nitllower” with the sequence: 5′-TGGAA(AG)TG(CT)TCCCA(AG)CA-3′,

Kobayashi, M., Komeda, H., Yanaka, N., Nagasawa, T. and Yamada, H. (1992) Nitrilase from Rhodococcus rhodochrous J1.

Kobayashi, M., Izui, H., Nagasawa, T. and Yamada, H. (1993) Nitrilase in biosynthesis of the plant hormone indole-3-acetic acid from indole-3-acetonitrile: Cloning of the Alcaligenes gene and site-directed mutagenesis of cysteine residues.

The sequence of the oligonucleotide was [lacuna] from a conserved amino acid sequence region with the presumed catalytic cysteine residue (Kobayashi et al., J. Biol. Chem. 267, 1992, 20746-20751 and Proc. Natl. Acad. Sci. USA, 90, 1993, 247-251). This motif was also found in the previously disclosed DNA sequences of the nitrilase gene from the strains Rhodococcus rhodochrous J1 (GenBank Acc. # D11425) and R.rhodochrous K22 (GenBank Acc. # D12583).

1.3.1 Transfer of DNA and hybridization

Round nylon membranes were placed on 5 agar plates with a total of 2500 plaques which had been prepared as described for determination of the titer in 1.2.2 for 1 min. The membranes were replaced-with plaque side on top on filter paper with denaturation solution (1.5 M NaCl, 0.5 M NaOH) for 2×5 min and then on filter paper with neutralization solution (0.5 M Tris/HCl, 1.5 M NaCl, pH 7.5) for 2×5 min. They were then briefly washed in 50 mM NaCl and dried, and the DNA was fixed at 120° C. for 30 min.

For the hybridization, the membranes were preincubated with 50 ml of hybridization buffer at 37° C. for 2 h and then hybridized with 10 pmol of ³²P-labeled oligonucleotide in 12 ml of hybridization buffer at 37° C. overnight. The oligonucleotide was labeled in a 30 μl mixture with 80 μCi of (γ-³²P)-ATP by 10 U of T4 polynucleotide kinase and separated from excess (γ-³²P)-ATP by drip column gel filtration with Sephadex G-25.

After the hybridization, the nylon membranes were washed with 0.5 g/l NaCl, 8.8 g/l Na citrate (2×SSC), 0.1% SDS at room temperature for 1×5 min and with 0.125 g/l NaCl, 2.2 g/l Na citrate (0.5×SSC), 0.1% SDS at 32° C. for 2×15 min, and exposed to an X-ray film in a film cassette with intensifying screen.

1.3.2 Identification and sequencing of the nitA gene

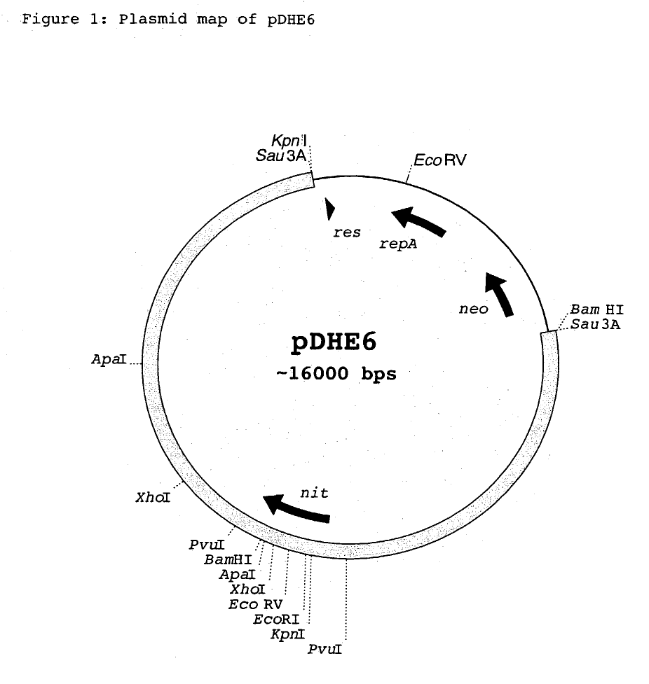

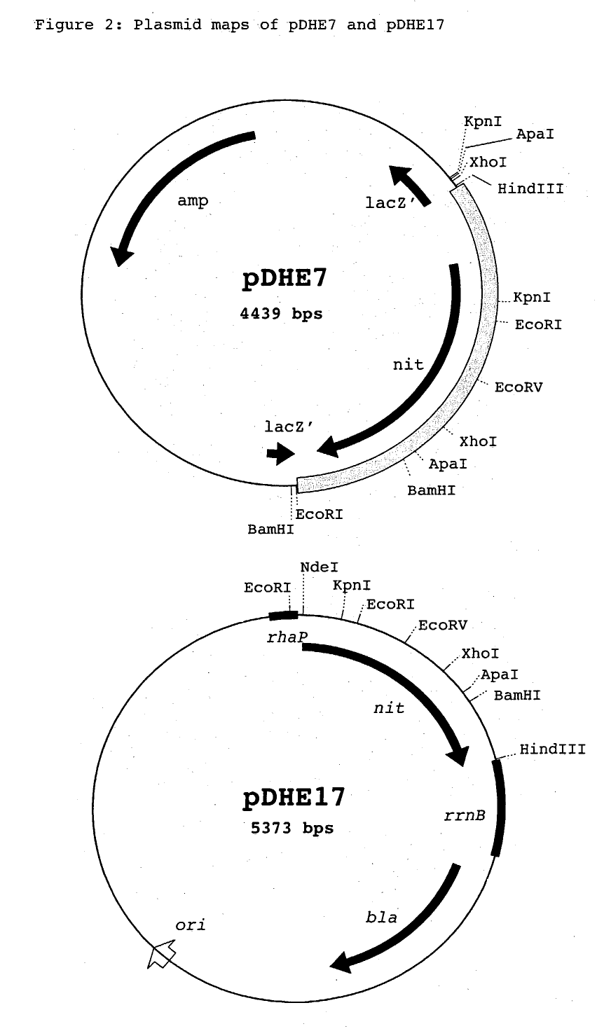

A total of 3 positive clones were identified, two of which harbored an incomplete nitA gene fragement and one harbored the complete nitA gene. The positive plaques were removed by stabbing, each incubated in 0.5 ml of SM buffer at room temperature for 2 h and, after adding 2 drops of chloroform, stored at 4° C. The plasmid resulting after conversion of the recombinant λ±RESIII phage with the complete nitA gene (see 1.2.3) was designated pDHE 6 (FIG. 1 shows pDHE. 6 with 12 kb of genomic gene bank fragment from Rhodococcus rhodochrous NCIMB 11216) and the vicinity of the nitA gene was restriction-mapped by Southern hybridizations using the oligonucleotide probe “nitllower” A 1.5 kb PvuI fragment with the complete nitA gene was treated with Klenow fragment and subcloned into EcoRV-treated pBluescriptSK+ (pDHE 7 with the 1.5 kb PvuI fragment from the genomic 12 kb gene bank fragment of Rhodococcus rhodochrous NCIMB 11216 in pDHE 6, FIG. 2). After further subcloning of the overlapping pDHE 7 fragments HindIII (vector)/EcoRI, KpnI/XhoI, EcoRV/BamHI and ApaI/EcoRI (vector) into pBluescriptSK+ correspondingly digested in each case, the PvuI fragment was subjected to double-stranded sequencing by the method of Sanger et al. (Proc. Natl. Acad. Sci. USA 74, 1977, 5463-5467) using an automatic sequencer. The sequencing reaction was carried out using a commercially available sequencing kit with the likewise commercially available universal and reverse primers (Vieira & Messing, Gene, 19, 1982: 259-268). The DNA sequence found for the 1.5 kb PvuI fragment is depicted in SEQ ID NO: 1. The derived amino acid sequence is to be found in SEQ ID NO: 2.

2 Heterologous expression of the nitA gene from R.rhodochrous NCIMB 11216 in E.coli and purification of the recombinant nitrilase protein

For cloning into an expression vector, the nitA gene from R.rhodochrous NCIMB 11216 was amplified from the translation start codon to the translation stop codon. The primers used for this were

“nit NdeI” (upper) with the sequence:

5′-TATATATCATATGGTCGAATACACAAACA-3′

and

“nit HindIII” (lower) with the sequence:

5′-TAATTAAGCTTCAGAGGGTGGCTGTCGC-3′

in which an NdeI cleavage site overlapping with the translation start is attached at the 5′-nitA end, and a HindIII cleavage site overlapping with the stop codon is attached at the 3′-nitA end. This pair of primers was used to amplify the nitA gene from pDHE 7 using Pwo polymerase in a reaction volume of 40 μl with in each case 8 pmol of primer, 100 pg of pDHE 7 template and 2.5 units of Pwo in 10 mM Tris/HCl, pH 8.85, 25 mM KCl, 5 mM (NH₄)SO₄ [sic], 2 mM MgSO₄, 0.2 mM dATP, 0.2 mM dTTP, 0.2 mM dGTP and 0.2 mM dCTP under the following conditions:

Denaturation at 94° C. for 3′;

25 cycles with denaturation at 93° C. for 1′, primer annealing at 48° C. for 1′30″ and polymerization at 72° C. for 1′30″;

Final polymerization at 72° C. for 5′.

The resulting nit PCR fragment was purified, digested with NdeI/HindIII and integrated into analogously digested molecules of the vector pJOE 2702 (Volff et al., Mol. Microbiol., 21, 1996: 1037-1047), and the resulting plasmid was designated pDHE 17 (FIG. 2: pDHE 17 with nitA in the L-rhamnose-inducible expression vector pJOE 2702). The integration via NdeI/HindIII means that the nitA gene in the plasmid pDHE 17 is under transcription control of the promoter rhaₚ which is present in pJOE 2702 and derives from the L-rhamnose operon rhaBAD in E.coli (Egan & Schleif, Mol. Biol. 243, 1994: 821-829). Termination of transcription of the nitA gene and initiation of translation of the transcripts likewise take place via vector sequences (Volff et al., 1996). After transformation of pDHE 17 into E.coli JM 109, the nitA gene from R.rhodochrous NCIMB 11216 can be induced by addition of L-rhamnose.

For purificatuion of the recombinant nitrilase protein by imidazole affinity chromatography, the nitA gene was additionally fused to a 3′ sequence for a C-terminal His₆ motif by using for amplification of the nitA gene, which took place under the conditions mentioned above, not only the 5′ primer “nitNdeI” (upper) but also a modified 3′ primer without stop codon having the sequence 5′-CGAGGGTGGCTGTCGCCCG-3′, and integrating the resulting PCR fragment in a modified pJOE 2702 vector which contained the sequence [CAT]₆TGA behind the BamHI cleavage site. BamHI digestion, Klenow treatment and NdeI digestion of the vector were followed by fusion of the nitA Pwo amplicon which had been cut with NdeI by ligation at the 3′ end through blunt ends in reading frame with the His₆ motif sequence, and the resulting plasmid was designated pDHE 18.

For heterologous expression on the laboratory scale, JM 109 (pDHE 17) from a 37° C. overnight culture was inoculated 1:200 in 50 ml dYT complete mediumn (Sambrook et al., 1989) with 0.2% L-rhamnose, and the culture was cultivated with induction in the shaking water bath at 30° C. for 8 h. The cells were then washed once in 50 mM Tris/HCl, pH 7.5, resuspended in the same buffer equivalent to an OD₆₀₀ of 10, and disrupted by ultrasound treatment. The procedure with JM 109 (pDHE 18) was analogous. The protein pattern of the crude extracts obtained by ultrasound treatment and clarified by centrifugation was determined by SDS polyacrylamide gel electrophoresis, comparing with the noninduced control; with the induction conditions mentioned, the proportion of nitrilase in the protein was about 30% for each of JM 109 (pDHE 17) and JM 109 (pDHE 18).

The nitrilase with His₆ motif from JM 109 (pDHE 18) was purified by washing the cells in 50 mM Tris/HCl, pH 7.5, resuspending equivalent to about 50 OD₆₀₀/ml and preparing extracts with a French press (2×at 20000 psi). Clarification of the extracts by centrifugation at 15000 g for 30 min was followed by purification with QIAexpress-Ni²⁺-NTA (QIAGEN). 1 ml of matrix equilibrated with 20 mM Tris/HCl, pH 7.5, was used per ml of crude extract. After loading of the column it was washed with 5 column volumes of 20 mM Tris/HCl, 300 mM NaCl, 40 mM imidazole, pH 7.0, and eluted with 20 mM Tris/HCl, 300 mM NaCl, 300 mM imidazole, pH 7.5. The purity of the nitrilase protein obtained in this way was >90% according to gel electrophoresis. After dialysis twice against 50 mM Tris/HCl, 0.1 mM DTT, 0.5 M (NH₄)₂SO₄, pH 7.5 it was possible to store the purified nitrilase at −20° C.

Measurements on the crude extracts showed in each case around 2 U/mg for the conversion of 2-benzonitrile [sic] into benzoic acid, and on the nitrilase with His₆ motif purified using QIAexpress-Ni²⁺-NTA showed around 11 U/mg at an enzyme concentration of 50 μg/ml. In this case, one unit is equivalent to the production of 1 μmol of benzoic acid at an initial benzonitrile concentration of 10 mM, 30° C. and pH 7.5. The conversions of 2-benzonitrile [sic] into benzoic acid via the nitrilase crude extract took place in 50 mM Tris/HCl, pH 7.5, and the conversions with purified nitrilase took place in 50 mM Tris/HCl, pH 7.5, 0.1 mM DTT. The formation of benzoic acid was determined by HPLC (RP18 column, 250×4 [lacuna], mobile phase 47% methanol, 0.3% H₃PO₄).

A number of nitriles were converted, and the conversions determined, in analogy to the example described above.

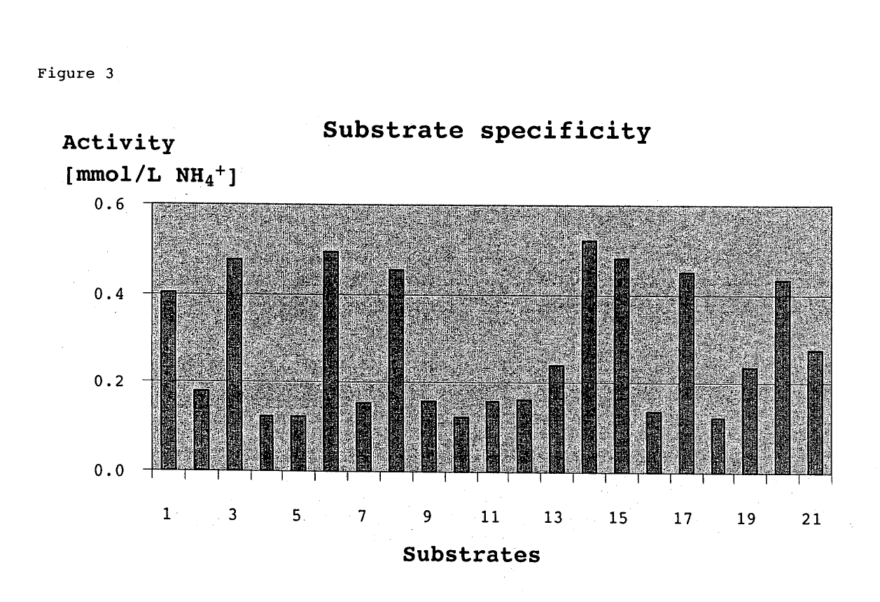

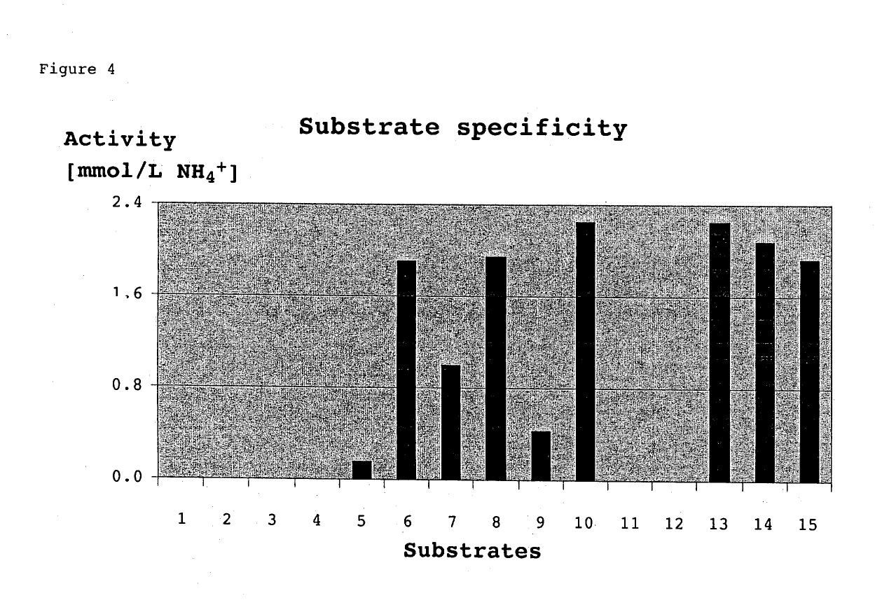

Various nitriles were converted using the E. coli strains JM 109 (pDHE 17 and pDHE 18). The cells were for this purpose cultured in 250 ml LB/Amp medium+2 g/l rhamnose at 30° C. and 200 rpm for 9 hours (=h). The cells were harvested by-centrifugation (20 min, 4° C., 5000 rpm). The cells were then resuspended in 10 mM phosphate buffer, pH 7.2, so that the concentration of dry biomass (DBM) was 2 g DBM/l. 150 μl portions of the cell suspension were pipetted into each well of a microtiter plate. The plate was then centrifuged. The supernatant was aspirated off and the cell pellets were washed twice with Na2HPO4 [sic] (1.42 g/lin Finnagua, pH 7.2). After another centrifugation step, the cell pellets are [sic] resuspended in the respective substrate solution (150 μl). One substrate was added to each row of 12 in the microtiter plate. A row with substrate solution but without cells was used as control (blank). The microtiter plates were incubated in a shaking incubator at 30° C. and 200 rpm for 1 h. The cells were then spun down and the amount of NH4 [sic] ions produced in the supernatant was determined using a Biomek instrument. Measurement took place at 620 nm, comparing with a calibration plot produced using various NH₄OH solutions. The substrates employed in Experiment 1 (see FIG. 3, Table 1) were the following substrates: benzonitrile (=1), 3-hydroxypropionitrile (=2), 2-methylglutaronitrile (=3), 4-chloro-3-hydroxybutyronitrile (=4), malononitrile (=5), crotononitrile (=6), geranonitrile (=7), octanedinitrile (=8), pivalonitrile (=9), aminocapronitrile (=10), 3,4-dihydroxybenzonitrile (=11), 3,5-dibromo-4-hydroxybenzonitrile (=12), 3-cyanopyridine (=13), 4-bromobenzyl cyanide (=14), 4-chlorobenzyl cyanide (=15), 2-phenylbutyronitrile (=16), 2-chlorobenzyl cyanide (=17), 2-pyridylacetonitrile (=18), 4-fluorobenzyl cyanide (=19), 4-methylbenzonitrile (=20), benzyl cyanide (=21). The substrates used in Experiment 2 (see FIG. 4, Table 2), which was carried out in analogy to Experiment 1, were as follows:

2-phenylpropionitrile (=1), mandelonitrile (=2),

2-amino-2-phenylacetonitrile (=3), 2-hydroxypropionitrile (=4),

3,3-dimethoxypropionitrile (=5), 3-cyanothiophene (=6),

3-cyanomethylthiophene (=7), benzonitrile (=8), propionitrile

(=9), trans-cinnamonitrile (=10), 2-hydroxy-4-phenylbutyronitrile

(=11), 3-phenylglutaronitrile (=12), fumaronitrile (=13),

glutaronitrile (=14) valeronitrile (=15).

TABLE 1

| | | |
|---|---|---|
| 1 | Benzonitrile | 0.4051 |
| 2 | 3-Hydroxypropionitrile | 0.1785 |
| 3 | 2-Methylglutaronitrile | 0.4758 |
| 4 | 4-Chloro-3-hydroxybutyronitrile | 0.1208 |
| 5 | Malononitrile | 0.1208 |
| 6 | Crotononitrile | 0.4946 |
| 7 | Geranonitrile | 0.1517 |
| 8 | Octanedinitrile | 0.4548 |
| 9 | Pivalonitrile | 0.1569 |
| 10 | Aminocapronitrile | 0.1236 |
| 11 | 3,4-Dihydroxybenzonitrile | 0.1569 |
| 12 | 3,5-Dibromo-4-hydroxybenzonitrile | 0.1624 |
| 13 | 3-Cyanopyridine | 0.2393 |
| 14 | 4-Bromobenzyl cyanide | 0.5213 |
| 15 | 4-Chlorobenzyl cyanide | 0.4830 |
| 16 | 2-Phenylbutyronitrile | 0.1376 |
| 17 | 2-Chlorobenzyl cyanide | 0.4530 |
| 18 | 2-Pyridylacetonitrile | 0.1222 |
| 19 | 4-Fluorobenzyl cyanide | 0.2361 |
| 20 | 4-Methylbenzonitrile | 0.4326 |
| 21 | Benzyl cyanide | 0.2755 |

TABLE 2

| | | |
|---|---|---|
| 1 | 2-Phenylpropionitrile | 0.0000 |
| 2 | Mandelonitrile | 0.0000 |
| 3 | 2-Amino-2-phenylacetonitrile | 0.0000 |
| 4 | 2-Hydroxypropionitrile | 0.0000 |
| 5 | 3,3-Dimethoxypropionitrile | 0.1466 |
| 6 | 3-Cyanothiophene | 1.9038 |
| 7 | 3-Cyanomethylthiophene | 0.9949 |
| 8 | Benzonitrile | 1.9518 |
| 9 | Propionitrile | 0.4135 |
| 10 | trans-cinnamonitrile | 2.2509 |
| 11 | 2-Hydroxy-4-phenylbutyronitrile | 0.0000 |
| 12 | 3-Phenylglutaronitrile | 0.0000 |
| 13 | Fumaronitrile | 2.2510 |
| 14 | Glutaronitrile | 2.0809 |
| 15 | Valeronitrile | 1.9218 |

We claim:

1. An isolated nucleic acid sequence which codes for a polypeptide having nitrilase activity, selected from the group of:
a) a nucleic acid sequence having the sequence depicted in SEQ ID NO: 1,
b) nucleic acid sequences which are derived from the nucleic acid sequence depicted in SEQ ID NO: 1 as a result of the degeneracy of the genetic code,
c) derivatives of the nucleic acid sequence depicted in SEQ ID NO: 1, which code for polypeptides having the amino acid sequences depicted in SEQ ID NO: 2 and have at least 97% homology at the amino acid level, with negligible reduction in the enzymatic action of the polypeptides.

2. An amino acid sequence encoded by a nucleic acid sequence as claimed in claim 1.

3. An amino acid sequence as claimed in claim 2, encoded by the sequence depicted in SEQ ID NO: 1.

4. A nucleic acid construct comprising a nucleic acid sequence as claimed in claim 1, the nucleic acid sequence being linked to one or more regulatory signals.

5. A vector comprising a nucleic acid sequence as claimed in claim 1 or a nucleic acid construct as claimed in claim 4.

6. A recombinant microorganism comprising a nucleic acid sequence as claimed in claim 1, a nucleic acid construct as claimed in claim 4, or a vector as claimed in claim 5.

7. A recombinant microorganism as claimed in claim 6, where the microorganism is a bacterium of the genera Escherichia, Rhodococcus, Nocardia, Streptomyces or Mycobacterium.

8. A process for preparing chiral or achiral carboxylic acids, which comprises converting nitrites in the presence of an amino acid sequence as claimed in claim 2 or 3 or a growing, dormant or disrupted microorganism as claimed in claim 6 or 7 into the chiral or achiral carboxylic acids.

9. A process for preparing chiral or achiral carboxylic acids as claimed in claim 8, wherein nitrites of the general formula I 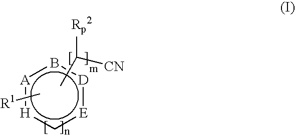
are converted in the presence of an amino acid sequence as claimed in claim 2 or 3 or a growing, dormant or disrupted microorganism as claimed in claim 6 or 7 into carboxylic acids of the general formula II 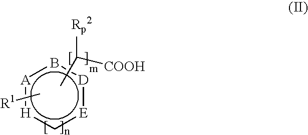
where the substituents and variables in the formulae I and II have the following meanings:
n=0 or 1
m=0, 1, 2 or 3, where for m>2 there is one or no double bond present between two adjacent carbon atoms,
p=0 or 1
A, B, D and E independently of one another are CH, N or CR³
H=O, S, NR⁴, CH or CR³, when n=0, or CH, N or CR³, when n =1,
it being possible for two adjacent variables A, B, D, E or H together to form another substituted or unsubstituted aromatic, saturated or partially saturated ring with 5 to 8 atoms in the ring which may contain one or more heteroatoms such as O, N or S, and not more than three of the variables A, B, D, E or H being a heteroatom,
R¹ is hydrogen, substituted or unsubstituted, branched or unbranched C₁-C₁₀-alkyl or C₁-C₁₀-alkoxy, substituted or unsubstituted aryl or hetaryl, hydroxyl, halogen, C₁-C₁₀-alkylamino or amino,
R² is hydrogen, substituted or unsubstituted, branched or unbranched C₁-C₁₀-alkyl or C₁-C₁₀-alkoxy, substituted or unsubstituted aryl or hetaryl, hydroxyl, C₁-C₁₀-alkylamino or amino,
R³ is hydrogen, substituted or unsubstituted, branched or unbranched C₁-C₁₀-alkyl or C₁-C₁₀-alkoxy, substituted or unsubstituted aryl, hetaryl, hydroxyl, halogen, C₁-C₁₀-alkylamino or amino,
R⁴ is hydrogen, substituted or unsubstituted, branched or unbranched C₁-C₁₀-alkyl.

10. A process as claimed in claim 8 or 9, wherein the process is carried out in an aqueous reaction solution at a pH between 4 and 11.

11. A process as claimed in any of claims 8 to 10, wherein from 0.01 to 10% by weight of nitrile are reacted in the process.

12. A process as claimed in any of claims 8 to 11, wherein the process is carried out at a temperature between 0° C. and 80° C.

13. A process as claimed in any of claims 8 to 12, wherein the achiral or chiral carboxylic acid is isolated from the reaction solution in yields of from 60 to 100% by extraction or crystallization or extraction and crystallization.
